(19) United States
(12) Patent Application Publication
(10) Pub. No.: US 2018/0136239 A1
(43) Pub. Date: May 17, 2018

(54) Diagnostic detection device and method for detecting human chorionic gonadotropin or derivative in urine, blood, saliva, or other body fluid

(71) Applicants: Zirui Deng, San Jose, CA (US); Weiwen Deng, San Jose, CA (US)

(72) Inventors: Zirui Deng, San Jose, CA (US); Weiwen Deng, San Jose, CA (US)

(21) Appl. No.: 15/352,596

(22) Filed: Nov. 16, 2016

(51) Int. Cl. G01N 33/76; G01N 33/543

(52) U.S. Cl. CPC G01N 33/76; G01N 33/54326; G01N 33/54346

(57) ABSTRACT

A diagnostic detection device and method with higher sensitivity to detect human chorionic gonadotropin (hCG) or derivative in urine, blood, saliva, or other body fluid for pregnancy test, detection of hCG doping in sports, or diagnosis of other diseases and conditions related to hCG abnormalities is disclosed. It harnesses the power of material capable of transporting specimen by capillary action, sandwich complex technique with a mobile anti-hCG antibody conjugated with a specific marker, hCG or derivative, and an immobile anti-hCG antibody, and a highly sensitive detection system for the marker or change of conditions of the marker. It may only comprise sandwich complex technique and detection system. It may simply comprise immobile anti-hCG antibody and detection system. It also can comprise immobile anti-hCG antibody and mobile standard hCG or derivative conjugated with a specific marker, and the latter competes against counterpart from specimen for a limited amount of anti-hCG antibody.

### BACKGROUND OF THE INVENTION

Chorionic gonadotropin (CG) is a gonadotropic glycoprotein hormone produced primarily by the placenta. Similar to the pituitary luteinizing hormone in structure and function, CG is involved in maintaining the corpus luteum during pregnancy. CG consists of two noncovalently linked subunits, alpha and beta. Within a species, the alpha subunit is virtually identical to the alpha subunits of the three pituitary glycoprotein hormones [thyroid-stimulating hormone (TSH), luteinizing hormone (LH), follicle-stimulating hormone (FSH)], but the beta subunit is unique and confers its biological specificity.

The structure of the beta subunit of human chorionic gonadotropin (hCG) is similar to the beta subunit of luteinizing hormone, except for the additional 30 amino acids at the carboxy end with the associated carbohydrate residues. HCG-beta is used as a diagnostic marker for early detection of pregnancy, spontaneous abortion, ectopic pregnancy, hydatiform mole, choriocarcinoma, or Down syndrome.

hCG test is conducted to check for hCG in blood or urine. HCG is made by placenta during pregnancy. An egg is normally fertilized by a sperm cell in a fallopian tube. Within 9 days after fertilization, the fertilized egg moves down the fallopian tube into the uterus and attaches, i.e. implants, to uterine wall. Once the fertilized egg implants, the developing placenta begins releasing hCG into blood, which usually happens 6 days after fertilization. Some hCG also gets passed in urine. hCG can be found in the blood before the first missed menstrual period, as early as 6 days after implantation.

hCG helps maintain pregnancy and affects development of fetus. Levels of hCG increase steadily in the first 14-16 weeks following a woman's last menstrual period (LMP), peak around the 14th week following LMP, and then decrease gradually. The amount that hCG increases early in pregnancy can give information about pregnancy and health of fetus. More hCG is released in a multiple pregnancy (e.g. twin or triplet) than in a single pregnancy. Less hCG is released if fertilized egg implants in a place other than the uterus (e.g. a fallopian tube), i.e. an ectopic pregnancy. After delivery, hCG cannot be found in blood

(http://www.webmd.com/baby/human-chorionic-gonadotropin-hcg).

hCG levels in blood:
1. Men and nonpregnant women: <5 international units per liter (IU/L)
2. Pregnant women, 1 week of gestation (about 3 weeks after the last menstrual period): 5-50 IU/L
3. Pregnant women, 2 weeks of gestation (about 4 weeks after the LMP): 50-500 IU/L
4. Pregnant women, 3 weeks of gestation (about 5 weeks after the LMP): 100-10,000 IU/L
5. Pregnant women, 4 weeks of gestation (about 6 weeks after the LMP): 1080-30,000 IU/L
6. Pregnant women, 6-8 weeks of gestation (about 8-10 weeks after the LMP): 3500-115,000 IU/L
7. Pregnant women, 12 weeks of gestation (about 14 weeks after the LMP): 12,000-270,000 IU/L
8. Pregnant women, 13-16 weeks of gestation (about 15-18 weeks after the LMP): Up to 200,000 IU/L

hCG levels in urine:
1. Men: None (negative test)
2. Nonpregnant women: None (negative test)
3. Pregnant women: Detectable (positive test)

If a woman is pregnant, very high levels of hCG can mean a multiple pregnancy (e.g. twins or triplets), a molar pregnancy, or Down syndrome. If a woman is pregnant, low levels of hCG can mean an ectopic pregnancy, or death of fetus. Levels of hCG that are decreasing abnormally in a pregnant woman may mean a miscarriage.

In a nonpregnant woman or a man, a high hCG level can mean a germ cell tumor (e.g. a tumor of testicle or ovary) is present. It may also mean cancer of stomach, pancreas, large intestine, liver, or lung.

Pregnancy tests are designed to tell if a woman's urine or blood contains hCG. Many women first choose hCG urine pregnancy test to take about a week after a missed period.

hCG blood pregnancy test can detect pregnancy earlier than hCG urine pregnancy test, or about 6-8 days after ovulation
(http://www.webmd.com/baby/guide/pregnancy-tests).

Levels of hCG currently can first be detected approximately 11 days following conception in a blood test. One to three days later, it can be detected in a urine test. Some highly sensitive tests can detect hCG as early as a week following ovulation. Normal home pregnancy tests are not able to detect hCG in the blood until at least 12 to 14 days after ovulation.

Most pregnancy tests employ a monoclonal antibody specific to the β-subunit of hCG (β-hCG). This procedure is employed to ensure that tests do not make false positives by confusing hCG with LH and FSH. The latter two are always present at varying levels in the body, whereas the presence of hCG almost always indicates pregnancy.

Many hCG immunoassays are based on the sandwich principle, which uses antibodies to hCG labeled with an enzyme or a conventional or luminescent dye. Pregnancy urine dipstick tests are based on the lateral flow technique. The urine pregnancy test may be a chromatographic immunoassay or any of several other test formats. Published detection thresholds range from 20 to 100 mIU/ml, depending on the brand of test. The blood pregnancy test is typically a chemiluminescent or fluorimetric immunoassay, or radioimmunoassay, that can detect β-hCG levels as low as 5 mIU/ml and allows quantification of β-hCG concentration.

In short, about 10 days after fertilized egg implants in uterus, urine hCG can be detected by antibody enzyme analysis. Minimum urine hCG level is 25 mIU/ml hCG for current antibody enzyme method. Therefore, the sensitivity of current urine or blood pregnancy test is low. If doing a pregnancy test for hCG very early in pregnancy (e.g. during the first week after implantation), the current test may not always show an early pregnancy, i.e. a false negative result.

Our purpose is to develop an improved pregnancy test with higher sensitivity to detect extremely low amount of hCG in urine, blood, or saliva of a pregnant woman, i.e. 20, 10, 5, 1, 0.1, 0.01 mIU/ml hCG or even lower. Thus pregnancy can be detected within 10, 5, 3, or even 1 day after fertilized egg implants onto the wall of uterine, i.e. many days before the day of missed period of a woman. Therefore, our novel pregnancy test device and method is more sensitive and can make a diagnosis of extremely early pregnancy.

Until the present invention, a need has existed to develop an improved pregnancy test to make a pregnancy diagnosis at an extremely early period of time so that the pregnant woman can know it right away. The present invention fulfills this need.

For doping in sports, hCG is banned by International Olympic Committee, mainly for its stimulation of testosterone production in testes. Therefore, the present invention can also be used to detect hCG or its derivative in urine, blood, saliva, or other body fluid of athletes.

For the diagnosis of other diseases and conditions related to hCG abnormalities, the present invention provides a better method with higher sensitivity.

### BRIEF SUMMARY OF THE INVENTION

A diagnostic detection device and method with improved sensitivity for human chorionic gonadotropin (hCG) or its derivative in urine, blood, saliva, or other body fluid for pregnancy test, detection of hCG doping in sports, or diagnosis of other health conditions and diseases related to hCG abnormalities is provided. The present invention encompasses an improved diagnostic device and method with higher sensitivity to detect hCG or its derivative in urine, blood, saliva, or other body fluid.

The device and method comprises materials capable of transporting specimen (e.g. urine, blood, saliva, or other body fluid) by capillary action, sandwich complex technique with a mobile anti-hCG antibody conjugated with a specific marker, hCG or its derivative, and an immobile anti-hCG antibody, and a highly sensitive detection system, i.e. device and method, for the specific marker or change of conditions of the specific marker. An immobile anti-mouse antibody may also be added into the device, i.e. a 2^{nd} way.

In a 3^{rd} way, it only comprise sandwich complex technique with a mobile or immobile anti-hCG antibody conjugated with a specific marker, hCG or its derivative, and an immobile or mobile anti-hCG antibody, and a highly sensitive detection system for the specific marker or change of conditions of the specific marker.

In a 4^{th} way, it simply comprise an immobile anti-hCG antibody conjugated with a specific marker and a highly sensitive detection system for the specific marker or change of conditions of the specific marker. For the 4^{th} way, an immobile anti-hCG antibody/specimen (which may or may not contain hCG or its derivative) conjugated with a specific marker, and a detection system can also be used.

In a 5^{th} way, it can comprise an immobile anti-hCG antibody and a mobile standard hCG or derivative conjugated with a specific marker, and the latter competes against counterpart from specimen (i.e. hCG or derivative from urine, blood, saliva, or other body fluid) for a limited amount of anti-hCG antibody. For the 5^{th} way, an immobile anti-hCG antibody/a mobile standard hCG or derivative/specimen (which may or may not contain hCG or its derivative) conjugated with a specific marker, and a detection system can also be used. Further, an immobile anti-hCG antibody, a mobile standard hCG or derivative conjugated with a specific marker 1, specimen (which may or may not contain hCG or its derivative) conjugated with a specific marker 2 can also be used. Marker 1 and 2 can be green and red. Relative intensities of competitive reaction of the two antigens to antibody, i.e. green or red signal, will be measured after washing off unbound hCG or its derivative. The red:green ratio will be used to quantify hCG or its derivative in specimen. Finally, a mobile anti-hCG antibody/an immobile standard hCG or derivative conjugated with a specific marker, and a detection system may also be used.

Therefore, a better diagnostic detection device and method with higher sensitivity to hCG or its derivative for testing early pregnancy, detecting hCG doping in sports, and diagnosing other health conditions and diseases related to hCG abnormalities is disclosed here.

hCG or derivative is beta subunit of hCG, hCG beta submit core fragment, hCG beta subunit derivative, alpha submit of hCG, hCG, or hCG derivative. In most situations, hCG or derivative is beta subunit of hCG.

An anti-hCG antibody is a mouse anti-hCG monoclonal antibody, a rabbit anti-hCG polyclonal antibody, a rabbit anti-hCG monoclonal antibody, a goat anti-hCG polyclonal antibody, or a sheep anti-hCG polyclonal antibody. All these anti-hCG antibodies are against beta subunit of hCG, hCG beta submit core fragment, hCG beta subunit derivative, alpha submit of hCG, hCG, or hCG derivative. In most situations, an anti-hCG antibody is an antibody to beta subunit of hCG.

In a preferred aspect, the device and method is composed of a variety of specific markers for high sensitivity detection as shown below:
a fluorochrome such as fluorescein [e.g. fluorescein isothiocyanate (FITC)], rhodamine, Texas Red, Cyanine Cy2, Cyanine Cy3, Cyanine Cy5, AMCA, phycoerythrin, and DyLight dyes;
an enzyme [e.g. horse radish peroxide (HRP), alkaline phosphatase (AP)];
a fluorochrome (e.g. a red/orange fluorescent dye, a green fluorescent dye, an aqua fluorescent dye) used in FISH to chromosome;
a fluorescent dye (e.g. a red fluorescent dye, a green fluorescent dye) used in array CGH or SNP array;
a gold nanoparticle (GNP), a silver nanoparticle, and other metal nanoparticle;
a quantum dot (QD);
a magnetic nanoparticle/magnetic bead;
a nano-bio-chip;
an encapsulated phase change nanoparticle;
a silica nanoparticle doped with fluorescence resonance energy transfer (FRET);
a carbon nanotube (CNT);
a nanowire, e.g. a silicon nanowire (SiNW), a gold nanowire, and a conducting polymer nanowire;
a microcantilever;
a nanopore, including synthetic, artificial, and protein-based nanopore;
a glucose;
a small molecular weight material.

In another aspect, the device and method comprises a highly sensitive detection system for each specific marker or change of conditions of each specific marker as shown below:
a common fluorescence image detection system;
a fluorescence detection method similar to that used in FISH to chromosome;
a method of imaging and analysis of fluorescence similar to that used in array CGH;
a method of imaging and analysis of fluorescence similar to that used in SNP array;
a smart phone scanning system for remote analysis of the marker;
an optical inspection system, optical sensor, photosensor;
enzyme/substrate reaction detection system;
nano magnet;
nano needle biosensor;
affinity based immunosensor;
electrical sensor;
quantum tunneling biosensor;
currency change detection system;
pH change detection system;
resistance change detection system;
wave change detector;
glucose sensor;
optoelectronic devices and methods for small molecular weight materials such as organic photoreceptors, organic photovoltaic devices (OPVs), and organic light-emitting diodes (OLEDs);
other devices and methods for detecting small molecular weight materials.

In a preferred aspect, our novel hCG diagnostic detection device (either combined as one device or separated as two devices) can also be connected to the internet through cable, Wi-Fi, or wireless networking to transmit signal or signal change of the specific marker or data obtained by the detection system to a remote place for detection, procession, and analysis. The communications can be through wireless transmission, cable connection, or optical communication with each other and also with a remote place.

Our device and method can be either at a regular size (like the urine or blood pregnancy test device currently at the market, or a well of a 96 well plate for ELISA analysis) or at a very small size (like a microarray chip currently used in array CGH or SNP array). For the latter, i.e. microarray model, very small amount of urine, blood, saliva, or other body fluid is needed. Novel microarray chips of miniature detection models containing tiny amount of a material capable of transporting specimen (e.g. urine, blood, saliva, or other body fluid) by capillary action, a mobile anti-hCG antibody conjugated with a specific marker, an immobile anti-hCG antibody, an immobile anti-mouse antibody, or a small well with sandwich complex, an immobile anti-hCG antibody, or an immobile anti-hCG antibody and a mobile marker labeled hCG or derivative, will be used. A microarray scanning device will also be used.

Therefore, a better diagnostic detection device and method with higher sensitivity for testing early pregnancy, detecting hCG doping in sports, diagnosing other diseases and conditions related to hCG abnormalities is disclosed here.

### BRIEF DESCRIPTION OF THE DRAWINGS

The foregoing summary, as well as the following detailed description of the invention, will be better understood when read in conjunction with the appended drawings. However, it should be understood that the invention is not limited to the precise arrangements and instrumentalities shown in the drawings, which are presently preferred.

FIG. 1 is schematic drawing of one model of our novel diagnostic detection device and method with higher sensitivity for pregnancy test, detection of hCG doping in sports, or diagnosis of other diseases and conditions related to hCG abnormalities. Ab1 is a mouse monoclonal antibody to a first epitope of beta subunit of hCG, hCG beta submit core fragment, hCG beta subunit derivative, alpha submit of hCG, hCG, or hCG derivative. Ab2 is a mouse anti-hCG monoclonal antibody, a rabbit anti-hCG polyclonal antibody, a rabbit anti-hCG monoclonal antibody, a goat anti-hCG polyclonal antibody, or a sheep anti-hCG polyclonal antibody. Ab2 binds a second epitope of beta subunit of hCG, hCG beta subunit core fragment, hCG beta subunit derivative, alpha submit of hCG, hCG, or hCG derivative. Thus, if hCG or its derivative (particularly hCG beta subunit) is present in urine, blood, saliva, or other body fluid, a sandwich complex comprising mobile Ab1-marker, hCG or its derivative (particularly hCG beta subunit), and immobile Ab2 will be formed in the center. The marker or change of conditions of the marker then can be detected in the center. If the device is at a microarray chip size, tiny amount of each material will be used and a microarray scanning device will also be used. This microarray model can be used for simultaneous analysis of multiple specimens or daily sequential analysis of specimen.

FIG. 2 is schematic drawing of another model of our novel diagnostic detection device and method with higher sensitivity for pregnancy test, detection of hCG doping in sports, or diagnosis of other diseases and conditions related to hCG abnormalities. Ab1 is a mouse monoclonal antibody to a first epitope of beta subunit of hCG, hCG beta submit core fragment, hCG beta subunit derivative, alpha submit of hCG, hCG, or hCG derivative. Ab2 is a mouse anti-hCG monoclonal antibody, a rabbit anti-hCG polyclonal antibody, a rabbit anti-hCG monoclonal antibody, a goat anti-hCG polyclonal antibody, or a sheep anti-hCG polyclonal antibody. Ab2 binds a second epitope of beta subunit of hCG, hCG beta subunit core fragment, hCG beta subunit derivative, alpha submit of hCG, hCG, or hCG derivative. Thus, if hCG or its derivative (particularly hCG beta subunit) is present in urine, blood, saliva, or other body fluid, a sandwich complex comprising mobile Ab1-marker, hCG or its derivative (particularly hCG beta subunit), and immobile Ab2 will be formed in the center. The marker or change of conditions of the marker then can be detected in the center. Ab3 is an anti-mouse Ig antibody. It usually recognizes both heavy and light chains (H+L) of mouse IgG or IgM. Ab3 is a horse anti-mouse IgG antibody, a goat anti-mouse IgG antibody, or a goat anti-mouse IgM antibody. Ab3 can bind Ab1 which is a mouse monoclonal antibody, usually mouse IgG or IgM. If there is an excess of mobile Ab1-marker bound with hCG or its derivative, or mobile Ab1-marker only, immobile Ab3 will bind mobile Ab1-marker and the marker or change of conditions of the marker can be detected in the top. If the urine, blood, saliva, or other body fluid does not contain hCG or its derivative, a sandwich complex will not be formed. Thus the marker cannot be detected in the center. However, immobile Ab3 will bind mobile Ab1-marker and the marker or change of conditions of the marker can be detected in the top. If the device is at a microarray chip size, tiny amount of each material will be used and a microarray scanning device will also be used. This microarray model can be used for simultaneous analysis of multiple specimens or daily sequential analysis of specimen.

FIG. 3 is schematic drawing of a 3^{rd} model of our novel diagnostic detection device and method with higher sensitivity for pregnancy test, detection of hCG doping in sports, or diagnosis of other diseases and conditions related to hCG abnormalities. Ab1 is a mouse monoclonal antibody to a first epitope of beta subunit of hCG, hCG beta submit core fragment, hCG beta subunit derivative, alpha submit of hCG, hCG, or hCG derivative. Ab2 is a mouse anti-hCG monoclonal antibody, a rabbit anti-hCG polyclonal antibody, a rabbit anti-hCG monoclonal antibody, a goat anti-hCG polyclonal antibody, or a sheep anti-hCG polyclonal antibody. Ab2 binds a second epitope of beta subunit of hCG, hCG beta subunit core fragment, hCG beta subunit derivative, alpha submit of hCG, hCG, or hCG derivative. Thus, if hCG or its derivative (particularly hCG beta subunit) is present in urine, blood, saliva, or other body fluid, a sandwich complex comprising mobile Ab1-marker, hCG or its derivative (particularly hCG beta subunit), and immobile Ab2 will be formed in the well. After rinse, the marker or change of conditions of the marker can be detected in the well. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens or daily sequential analysis of specimen. For this method, a mobile anti-hCG antibody conjugated with a specific marker/an immobile anti-hCG antibody, or an immobile anti-hCG antibody conjugated with a specific marker/a mobile anti-hCG antibody can be used.

FIG. 4 is schematic drawing of a 4^{th} model of our novel diagnostic detection device and method with higher sensitivity for pregnancy test, detection of hCG doping in sports, or diagnosis of other diseases and conditions related to hCG abnormalities. Ab1 is a mouse monoclonal antibody to beta subunit of hCG, hCG beta submit core fragment, hCG beta subunit derivative, alpha submit of hCG, hCG, or hCG derivative. If hCG or its derivative (particularly hCG beta subunit) is present in urine, blood, saliva, or other body fluid, it will bind immobile Ab1-marker, and change of conditions of the marker may be detected in the well. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens or daily sequential analysis of specimen. For this method, an immobile anti-hCG antibody conjugated with a specific marker, or an immobile anti-hCG antibody/specimen (which may or may not contain hCG or its derivative) conjugated with a specific marker may be used.

FIG. 5 is schematic drawing of a 5^{th} model of our novel diagnostic detection device and method with higher sensitivity for pregnancy test, detection of hCG doping in sports, or diagnosis of other diseases and conditions related to hCG abnormalities. Ab1 is a mouse monoclonal antibody to beta subunit of hCG, hCG beta submit core fragment, hCG beta subunit derivative, alpha submit of hCG, hCG, or hCG derivative. Standard hCG or its derivative (particularly hCG beta subunit) is used to make the mobile hCG or its derivative-marker. If hCG or its derivative (particularly hCG beta subunit) is present in urine, blood, saliva, or other body fluid, it will compete against mobile hCG or its derivative-marker for a limited amount of immobile Ab1. After rinse, the marker or change of conditions of the marker can be detected in the well. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens or daily sequential analysis of specimen. For this method, an immobile anti-hCG antibody/a mobile standard hCG or derivative conjugated with a specific marker, an immobile anti-hCG antibody/a mobile standard hCG or derivative/specimen (which may or may not contain hCG or its derivative) conjugated with a specific marker, an immobile anti-hCG antibody/a mobile standard hCG or derivative conjugated with a specific marker 1/specimen (which may or may not contain hCG or its derivative) conjugated with a specific marker 2, or a mobile anti-hCG antibody/an immobile standard hCG or derivative conjugated with a specific marker may be used.

### DETAILED DESCRIPTION OF THE INVENTION

Human chorionic gonadotropin (hCG) is a hormone produced by syncytiotrophoblast, a portion of placenta following implantation. The presence of hCG in blood or urine is detected in pregnancy tests. Blood or urine tests measure hCG for pregnancy tests. hCG-positive indicates an implanted blastocyst and embryogenesis. As pregnancy tests, blood test and the most sensitive urine test usually detect hCG 6-12 days after ovulation.

Some tumors produce hCG. Therefore, elevated levels measured when the patient is not pregnant can lead to a cancer diagnosis. Blood or urine hCG tests can be conducted to diagnose and monitor germ cell tumors and gestational trophoblastic diseases. Thus, hCG can be used as a tumor marker. The β subunit of hCG is secreted by some tumors including seminoma, choriocarcinoma, germ cell tumors, teratoma with elements of choriocarcinoma, and islet cell tumor. A positive hCG result in a male can be a test for testicular cancer. The normal hCG range for men is between 0-5 mIU/mL. Gestational trophoblastic disease like hydatidiform moles (molar pregnancy) or choriocarcinoma may produce high levels of β-hCG, due to presence of syncytialtrophoblast-a part of the placenta.

In short, besides detecting pregnancy and hCG doping in sports, measurement of hCG is useful in monitoring germ cell and trophoblastic tumors, follow-up care after miscarriage, and in diagnosis of and follow-up care after treatment of ectopic pregnancy. Lack of a visible fetus on vaginal ultrasound after β hCG levels have reached 1500 mIU/ml may be strongly indicative of an ectopic pregnancy.

The invention herein described demonstrates another way to better detect hCG or its derivative in urine, blood, saliva, or other body fluid for pregnancy test or detection of hCG doping in sports. The method can also be used to detect ectopic pregnancy, spontaneous abortion, hydatiform mole, choriocarcinoma, germ cell tumors, testicular cancer, seminoma, teratoma with elements of choriocarcinoma, islet cell tumor, and Down syndrome, which are all related to hCG abnormalities.

The invention comprises and utilizes specific markers and high sensitive detection systems for the marker and change of conditions of the marker, thereby improving sensitivity to detect hCG or its derivative in urine, blood, saliva, or other body fluid.

The invention includes a device and method of better detection of hCG or its derivative in urine, blood, saliva, or other body fluid for pregnancy test, detection of hCG doping in sports, or diagnosis of other diseases and conditions related to hCG abnormalities. This device and method comprises a material capable of transporting urine, blood, or saliva by capillary action, a mobile anti-hCG antibody conjugated with a specific marker, an immobile anti-hCG antibody, and a detection system for the marker or change of conditions of the marker. An immobile anti-mouse antibody may also be added into the device.

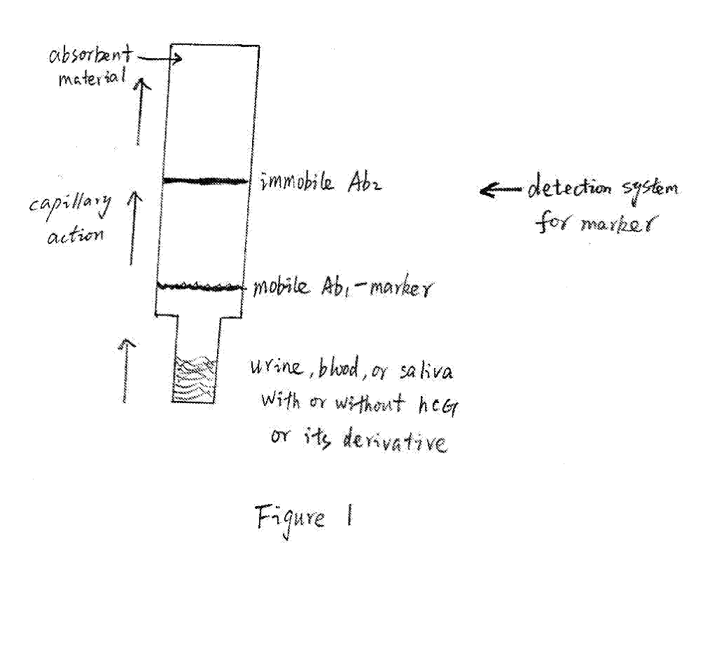

Generally, a material capable of transporting specimen (e.g. urine, blood, saliva, or other body fluid) by capillary action is a test dipstick of absorbent material, i.e. absorbent material in FIG. 1, usually inside a plastic. Near the bottom tip, it is coated with a mobile anti-hCG antibody labeled with a specific marker for detection, i.e. mobile Ab1-marker in FIG. 1. This mobile anti-hCG antibody can bind hCG or its derivative. In the center, it is coated with an immobile anti-hCG antibody, i.e. immobile Ab2 in FIG. 1. This immobile anti-hCG antibody can bind hCG or its derivative that is already bound to the mobile anti-hCG antibody labeled with a marker. When the absorbent bottom tip dipped in specimen or when dropping specimen onto the bottom tip, specimen migrates via capillary action carrying the marker labeled mobile anti-hCG antibody, i.e. mobile Ab1-marker in FIG. 1, up to the center where the immobile anti-hCG antibody, i.e. immobile Ab2 in FIG. 1, is located. If urine, blood, saliva, or other body fluid contains hCG or its derivative (particularly hCG beta subunit), hCG or its derivative will migrate up, first bind the mobile anti-hCG antibody labeled with a marker, migrate up again, and then a sandwich complex containing marker labeled mobile anti-hCG antibody, hCG or its derivative (particularly hCG beta subunit), and immobile anti-hCG antibody, i.e. mobile Ab1-marker/hCG or its derivative/immobile Ab2, will be formed in the center. Thus the marker or change of conditions of the marker can be detected in the center by a detection system, i.e. positive for pregnancy, hCG doping in sports, or other diseases and conditions related to hCG abnormalities. If the urine, blood, saliva, or other body fluid does not contain hCG or its derivative, a sandwich complex will not be formed, and marker labeled mobile anti-hCG antibody, i.e. mobile Ab1-marker in FIG. 1, will keep migrating beyond the center and up to the top. Thus the marker cannot be detected in the center by a detection system, i.e. negative for pregnancy, hCG doping in sports, or other diseases and conditions related to hCG abnormalities. See FIG. 1 for more information. If the device is at a microarray chip size, tiny amount of each material will be used and a microarray scanning device will also be used. This microarray model can be used for simultaneous analysis of multiple specimens (e.g. 5, 25, hundreds or even more specimens) or daily sequential analysis of specimen (e.g. 5, 25, 365 or even more days).

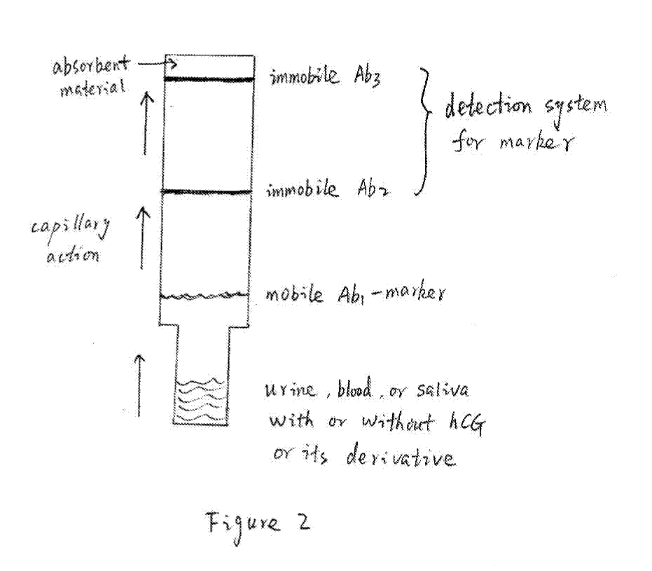

Furthermore, a material capable of transporting specimen (e.g. urine, blood, saliva, or other body fluid) by capillary action is a test dipstick of absorbent material, i.e. absorbent material in FIG. 2, usually inside a plastic. Near the bottom tip, it is coated with a mobile anti-hCG antibody labeled with a specific marker for detection, i.e. mobile Ab1-marker in FIG. 2. This mobile anti-hCG antibody can bind hCG or its derivative. In the center, it is coated with an immobile anti-hCG antibody, i.e. immobile Ab2 in FIG. 2. This immobile anti-hCG antibody can bind hCG or its derivative that is already bound to the mobile anti-hCG antibody labeled with a marker. In the top, it is coated with an immobile anti-mouse antibody, i.e. immobile Ab3 in FIG. 2. This immobile anti-mouse antibody can bind the mobile anti-hCG antibody labeled with a marker, no matter whether it has been bound to hCG or its derivative or not. When the absorbent bottom tip dipped in specimen or when dropping specimen onto the bottom tip, specimen migrates via capillary action carrying the marker labeled mobile anti-hCG antibody, i.e. mobile Ab1-marker in FIG. 2, up to the center where the immobile anti-hCG antibody, i.e. immobile Ab2 in FIG. 2, is located. If urine, blood, saliva, or other body fluid contains hCG or its derivative (particularly hCG beta subunit), hCG or its derivative will migrate up, first bind the mobile anti-hCG antibody labeled with a marker, migrate up again, and then a sandwich complex containing marker labeled mobile anti-hCG antibody, hCG or its derivative (particularly hCG beta subunit), and immobile anti-hCG antibody, i.e. mobile Ab1-marker/hCG or its derivative/immobile Ab2, will be formed in the center. Thus the marker or change of conditions of the marker can be detected in the center by a detection system, i.e. positive for pregnancy, hCG doping in sports, or other diseases and conditions related to hCG abnormalities. If there is an excess of marker labeled mobile anti-hCG antibody bound with hCG or its derivative, or marker labeled mobile anti-hCG antibody only, it will keep migrating beyond the center and up to the top, where the immobile anti-mouse antibody, i.e. immobile Ab3 in FIG. 2, is located. The immobile anti-mouse antibody will bind marker labeled mobile anti-hCG antibody, i.e. mobile Ab1-marker, and the marker or change of conditions of the marker can be detected in the top by a detection system. If the urine, blood, saliva, or other body fluid does not contain hCG or its derivative, a sandwich complex will not be formed. Thus the marker cannot be detected in the center by a detection system, i.e. negative for pregnancy, hCG doping in sports, or other diseases and conditions related to hCG abnormalities. The marker labeled mobile anti-hCG antibody, i.e. mobile Ab1-marker in FIG. 2, will keep migrating beyond the center and up to the top, where the immobile anti-mouse antibody, i.e. immobile Ab3 in FIG. 2, is located. The immobile anti-mouse antibody will bind marker labeled mobile anti-hCG antibody and the marker can be detected in the top by a detection system, i.e. test functions properly and control is good. See FIG. 2 for more information. If the device is at a microarray chip size, tiny amount of each material will be used and a microarray scanning device will also be used. This microarray model can be used for simultaneous analysis of multiple specimens (e.g. 5, 25, hundreds or even more specimens) or daily sequential analysis of specimen (e.g. 5, 25, 365 or even more days).

The material capable of transporting urine, blood, saliva, or other body fluid by capillary action is any absorbent material capable of transporting an aqueous solution including urine, blood, saliva, or other body fluid by capillary action, wicking, or simple wetting such as a paper towel, a nitrocellulose membrane, a glass fiber, and a capillary tube made of glass or any other substance that is polar.

The mobile anti-hCG antibody conjugated with a specific marker is an anti-hCG antibody which binds a first epitope of hCG or its derivative. The antibody is deposited near the bottom of the dipstick and can migrate up to the center or the top along with urine, blood, saliva, or other body fluid through capillary action. Thus it is mobile. The mobile anti-hCG antibody is a mouse monoclonal antibody to a first epitope of beta subunit of hCG, hCG beta submit core fragment, hCG beta subunit derivative, alpha submit of hCG, hCG, or hCG derivative.

The immobile anti-hCG antibody is an antibody which binds a second epitope of hCG or its derivative, so that if hCG or its derivative is present in urine, blood, saliva, or other body fluid, a sandwich complex comprising mobile anti-hCG antibody conjugated with the marker, hCG or its derivative, and immobile anti-hCG antibody is formed. The antibody is located in the center of the dipstick and cannot migrate up along with urine, blood, saliva, or other body fluid through capillary action. Thus it is immobile. There are many methods to immobilize anti-hCG antibody on absorbent material of dipstick. For example, biotin can be used to label the immobile anti-hCG antibody. Streptavidin or polymerized streptavidin is then used to immobilize this biotinylated antibody to the dipstick. The biotin-streptavidin interaction is strong. The binding between streptavidin or polymerized streptavidin and absorbent material must also be strong for a high efficient immobilization. The immobile anti-hCG antibody is a mouse anti-hCG monoclonal antibody, a rabbit anti-hCG polyclonal antibody, a rabbit anti-hCG monoclonal antibody, a goat anti-hCG polyclonal antibody, or a sheep anti-hCG polyclonal antibody. It binds a second epitope of beta subunit of hCG, hCG beta subunit core fragment, hCG beta subunit derivative, alpha submit of hCG, hCG, or hCG derivative.

The immobile anti-mouse antibody is an anti-mouse immunoglobulin (Ig) antibody. It usually recognizes both heavy and light chains (H+L) of mouse IgG or IgM. Examples are: a horse anti-mouse IgG antibody, a goat anti-mouse IgG antibody, and a goat anti-mouse IgM antibody. The antibody is located in the top of the dipstick and cannot migrate up along with urine, blood, saliva, or other body fluid through capillary action. Thus it is immobile. There are many methods to immobilize anti-mouse antibody on absorbent material of dipstick. Biotin-streptavidin/polymerized streptavidin is one example.

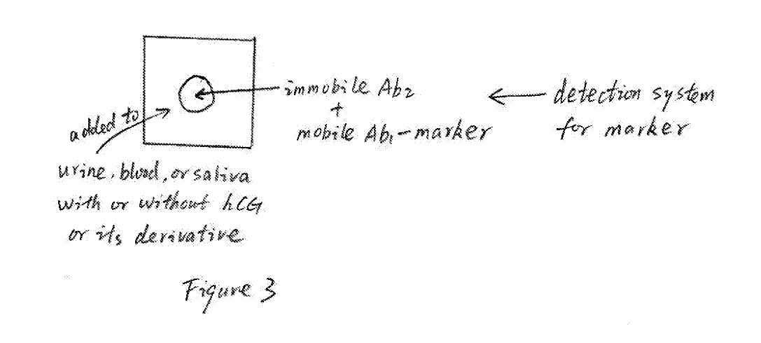

In a 3^{rd} way, a small well or spot (which is made of treated glass, nitrocellulose or nylon membrane, or other materials) is coated with an immobile anti-hCG antibody and a mobile anti-hCG antibody labeled with a specific marker for detection, i.e. immobile Ab2 and mobile Ab1-marker in FIG. 3. The mobile anti-hCG antibody can bind hCG or its derivative. The immobile anti-hCG antibody can bind hCG or its derivative that is already bound to the mobile anti-hCG antibody labeled with a marker. Urine, blood, saliva, or other body fluid will then be added to the well. If urine, blood, saliva, or other body fluid contains hCG or its derivative (particularly hCG beta subunit), a sandwich complex containing marker labeled mobile anti-hCG antibody, hCG or its derivative (particularly hCG beta subunit), and immobile anti-hCG antibody, i.e. mobile Ab1-marker/hCG or its derivative/immobile Ab2, will be formed in the well. After rinse, the marker or change of conditions of the marker can be detected in the well by a detection system, i.e. positive for pregnancy, hCG doping in sports, or other diseases and conditions related to hCG abnormalities. If the urine, blood, saliva, or other body fluid does not contain hCG or its derivative, a sandwich complex will not be formed, and marker labeled mobile anti-hCG antibody, i.e. mobile Ab1-marker in FIG. 3, will be removed by rinse. Thus the marker cannot be detected in the well by a detection system, i.e. negative for pregnancy, hCG doping in sports, or other diseases and conditions related to hCG abnormalities. See FIG. 3 for more information. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens (e.g. 5, 25, hundreds or even more specimens) or daily sequential analysis of specimen (e.g. 5, 25, 365 or even more days). For this method, an immobile anti-hCG antibody conjugated with a specific marker and a mobile anti-hCG antibody can also be used.

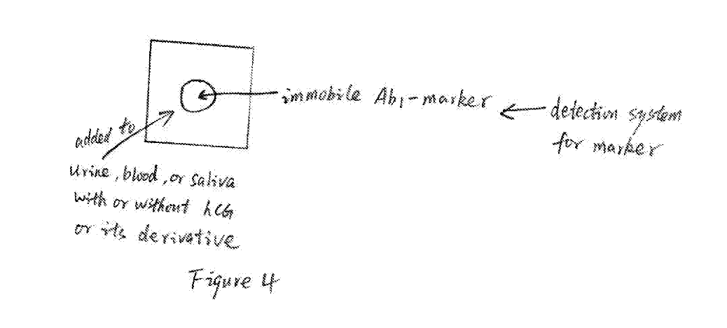

In a 4^{th} way, a small well or spot (which is made of treated glass, nitrocellulose or nylon membrane, or other materials) is coated with an immobile anti-hCG antibody labeled with a specific marker for detection, i.e. immobile Ab1-marker in FIG. 4. Urine, blood, saliva, or other body fluid will then be added to the well. If urine, blood, saliva, or other body fluid contains hCG or its derivative (particularly hCG beta subunit), it will bind immobile anti-hCG antibody labeled with a specific marker, i.e. immobile Ab1-marker/hCG or its derivative will be formed in the well. Change of conditions of the marker may be detected in the well by a detection system, i.e. positive for pregnancy, hCG doping in sports, or other diseases and conditions related to hCG abnormalities. If urine, blood, saliva, or other body fluid does not contain hCG or its derivative, no antigen and antibody reaction will happen. Thus change of conditions of the marker cannot be detected in the well by a detection system, i.e. negative for pregnancy, hCG doping in sports, or other diseases and conditions related to hCG abnormalities. See FIG. 4 for more information. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens (e.g. 5, 25, hundreds or even more specimens) or daily sequential analysis of specimen (e.g. 5, 25, 365 or even more days). For this method, an immobile anti-hCG antibody and specimen (which may or may not contain hCG or its derivative) conjugated with a specific marker may also be used.

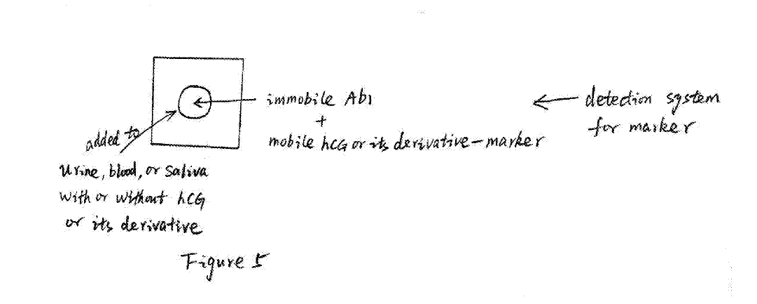

In a 5^{th} way, a small well or spot (which is made of treated glass, nitrocellulose or nylon membrane, or other materials) is coated with an immobile anti-hCG antibody and a mobile standard hCG or its derivative (particularly hCG beta subunit) labeled with a specific marker for detection, i.e. immobile Ab1 and mobile hCG or its derivative-marker in FIG. 5. Urine, blood, saliva, or other body fluid will then be added to the well. If hCG or its derivative (particularly hCG beta subunit) is present in urine, blood, saliva, or other body fluid, it will compete against mobile hCG or its derivative-marker for a limited amount of immobile Ab1 in the well, i.e. immobile Ab1/mobile hCG or its derivative-marker v. immobile Ab1/hCG or its derivative. After rinse, the marker at a decreased level or change of conditions of the marker can be detected in the well by a detection system, i.e. positive for pregnancy, hCG doping in sports, or other diseases and conditions related to hCG abnormalities. If urine, blood, saliva, or other body fluid does not contain hCG or its derivative, only mobile hCG or its derivative-marker binds immobile Ab1 in the well, i.e. immobile Ab1/mobile hCG or its derivative-marker. After rinse, the marker can be detected at the highest level or no change of conditions of the marker in the well by a detection system, i.e. negative for pregnancy, hCG doping in sports, or other diseases and conditions related to hCG abnormalities. See FIG. 5 for more information. The device can be either at a regular size (like a well of a 96 well plate for ELISA analysis) or at a very small size (like a microarray chip currently used in array CGH or SNP array). For the latter, i.e. microarray model, tiny amount of each material will be used and a microarray scanning device will also be used. This microarray model can be used for simultaneous analysis of multiple specimens (e.g. 5, 25, hundreds or even more specimens, like array CGH analysis) or daily sequential analysis of specimen (e.g. 5, 25, 365 or even more days). For this method, an immobile anti-hCG antibody/a mobile standard hCG or derivative/specimen (which may or may not contain hCG or its derivative) conjugated with a specific marker, or a mobile anti-hCG antibody and an immobile standard hCG or derivative conjugated with a specific marker may also be used. Further, an immobile anti-hCG antibody/a mobile standard hCG or derivative conjugated with a specific marker 1/specimen (which may or may not contain hCG or its derivative) conjugated with a specific marker 2 can also be used. Marker 1 and 2 can be green and red. Relative intensities of competitive reaction of the two antigens to antibody, i.e. green or red signal, will be measured after washing off unbound hCG or its derivative. The red:green ratio will be used to quantify hCG or its derivative in specimen, which is similar to what is used in array CGH or SNP array.

The specific marker is a fluorochrome such as fluorescein [e.g. fluorescein isothiocyanate (FITC)], rhodamine, Texas Red, Cyanine Cy2, Cyanine Cy3, Cyanine Cy5, AMCA, phycoerythrin, and DyLight dyes, used in a way similar to the way used in immunofluorescence staining of cell or tissue.

The specific marker is an enzyme [e.g. horse radish peroxide (HRP), alkaline phosphatase (AP)], used in a way similar to the way used in immunocytochemistry or immunohistochemistry of cell or tissue.

The specific marker is a fluorochrome (e.g. a red/orange fluorescent dye, a green fluorescent dye, an aqua fluorescent dye), used in a way similar to the way used in fluorescence in situ hybridization (FISH) to chromosome.

The specific marker is a fluorescent dye (e.g. a red fluorescent dye, a green fluorescent dye), used in a way similar to the way used in chromosome and genome analysis using microarrays such as microarray-based comparative genome hybridization (CGH), i.e. array CGH.

The specific marker is a fluorescent dye (e.g. a red fluorescent dye, a green fluorescent dye), used in a way similar to the way used in chromosome and genome analysis using microarrays such as single nucleotide polymorphism (SNP) array.

The specific marker is a nanomaterial, e.g. a gold nanoparticle (GNP), a silver nanoparticle, and other metal nanoparticle.

The specific marker is a nanomaterial, e.g. a quantum dot (QD).

The specific marker is a nanomaterial, e.g. a magnetic nanoparticle/magnetic bead.

The specific marker is a nanomaterial, e.g. a nano-bio-chip.

The specific marker is a nanomaterial, e.g. an encapsulated phase change nanoparticle.

The specific marker is a nanomaterial, e.g. a silica nanoparticle doped with fluorescence resonance energy transfer (FRET).

The specific marker is a nanomaterial, e.g. a carbon nanotube (CNT).

The specific marker is a nanomaterial, e.g. a nanowire, such as a silicon nanowire (SiNW), a gold nanowire, and a conducting polymer nanowire.

The specific marker is a microcantilever. Microfabricated cantilevers bend according to changes in environment or changes on their surface, and this bending is in the nano-meter scale.

The specific marker is a nanomaterial, e.g. a nanopore, including synthetic, artificial, and protein-based nanopore.

The specific marker is a glucose.

The specific marker is a small molecular weight material.

The detection system for the marker is a corresponding detection system used to detect the specific marker respectively. It is a common fluorescence image detection system, a fluorescence detection method similar to that used in FISH to chromosome, a method of imaging and analysis of fluorescence similar to that used in array CGH, a method of imaging and analysis of fluorescence similar to that used in SNP array, a smart phone scanning system for remote analysis of the marker, an optical inspection system, optical sensor, photosensor, enzyme/substrate reaction, nano magnet, nano needle biosensor, affinity based immunosensor, electrical sensor, quantum tunneling biosensor, currency change detector, pH change detection system, resistance change detection system, wave change detector, glucose sensor, optoelectronic devices and methods for small molecular weight materials such as organic photoreceptors, organic photovoltaic devices (OPVs), and organic light-emitting diodes (OLEDs), and other devices and methods for detecting small molecular weight materials.

This detection system for the marker can be inside the diagnostic detection device containing a material capable of transporting specimen by capillary action, a mobile anti-hCG antibody conjugated with a specific marker, an immobile anti-hCG antibody, and an immobile anti-mouse antibody or the diagnostic detection device containing a small well with sandwich complex, an immobile anti-hCG antibody, or an immobile anti-hCG antibody and a mobile marker labeled hCG or derivative. This detection system for the marker can also be a separate device from the diagnostic detection device containing a material capable of transporting specimen by capillary action, a mobile anti-hCG antibody conjugated with a specific marker, an immobile anti-hCG antibody, and an immobile anti-mouse antibody, or the diagnostic detection device containing a small well with sandwich complex, an immobile anti-hCG antibody, or an immobile anti-hCG antibody and a mobile marker labeled hCG or derivative.

Our novel diagnostic detection device and method containing a material capable of transporting specimen (e.g. urine, blood, saliva, or other body fluid) by capillary action, a mobile anti-hCG antibody conjugated with a specific marker, an immobile anti-hCG antibody, an immobile anti-mouse antibody, and a highly sensitive detection system for the specific marker or change of conditions of the specific marker, or a small well with sandwich complex, an immobile anti-hCG antibody conjugated with a specific marker, an immobile anti-hCG antibody/specimen (which may or may not contain hCG or its derivative) conjugated with a specific marker, or an immobile anti-hCG antibody/a mobile standard hCG or derivative conjugated with a specific marker, an immobile anti-hCG antibody/a mobile standard hCG or derivative/specimen (which may or may not contain hCG or its derivative) conjugated with a specific marker, an immobile anti-hCG antibody/a mobile standard hCG or derivative conjugated with a specific marker 1/specimen (which may or may not contain hCG or its derivative) conjugated with a specific marker 2, a mobile anti-hCG antibody/an immobile standard hCG or derivative conjugated with a specific marker, either as one device or separating the detection system from the others as two devices, can also be connected to the internet through cable, Wi-Fi, or wireless networking to transmit signal or signal change of the specific marker or data obtained by the detection system to a remote place for detection, procession, and analysis. The communications can be through wireless transmission, cable connection, or optical communication with each other and also with a remote place.

Our device and method can be either at a regular size (like the urine or blood pregnancy test device currently at the market, or a well of a 96 well plate for ELISA analysis) or at a very small size (like a microarray chip currently used in array CGH or SNP array). For the latter, i.e. microarray model, very small amount of urine, blood, saliva, or other body fluid is needed. Novel microarray chips of miniature detection models containing tiny amount of a material capable of transporting specimen (e.g. urine, blood, saliva, or other body fluid) by capillary action, a mobile anti-hCG antibody conjugated with a specific marker, an immobile anti-hCG antibody, an immobile anti-mouse antibody, or a tiny well with sandwich complex (i.e. an immobile or mobile anti-hCG antibody, and mobile or immobile hCG or derivative conjugated with a specific marker), an immobile anti-hCG antibody conjugated with a specific marker, an immobile anti-hCG antibody/specimen (which may or may not contain hCG or its derivative) conjugated with a specific marker, or an immobile anti-hCG antibody/mobile standard hCG or derivative conjugated with a specific marker, an immobile anti-hCG antibody/a mobile standard hCG or derivative/specimen (which may or may not contain hCG or its derivative) conjugated with a specific marker, an immobile anti-hCG antibody/a mobile standard hCG or derivative conjugated with a specific marker 1/specimen (which may or may not contain hCG or its derivative) conjugated with a specific marker 2, a mobile anti-hCG antibody/an immobile standard hCG or derivative conjugated with a specific marker, will be used. A novel microarray scanning device will also be used to detect signal or signal change of the specific marker.

In conclusion, a diagnostic detection device and method with improved sensitivity for hCG or its derivative in urine, blood, saliva, or other body fluid for pregnancy test, detection of hCG doping in sports, or diagnosis of other diseases and conditions related to hCG abnormalities is provided here. The invention encompasses an improved diagnostic device and method with higher sensitivity to detect hCG or its derivative in urine, blood, saliva, or other body fluid. It harnesses the power of materials capable of transporting urine, blood, saliva, or other body fluid by capillary action, sandwich complex technique with a mobile anti-hCG antibody conjugated with a specific marker, hCG or its derivative, and an immobile anti-hCG antibody, and high sensitive detection systems, i.e. devices and methods, for the marker or change of conditions of the marker. It may only comprise sandwich complex technique and detection system. It may simply comprise immobile anti-hCG antibody and detection system. It also can comprise an immobile anti-hCG antibody and a mobile standard hCG or derivative conjugated with a specific marker, and the latter competes against counterpart from urine, blood, saliva, or other body fluid for a limited amount of anti-hCG antibody. Therefore, a better diagnostic detection device and method with higher sensitivity for testing early pregnancy, detecting hCG doping in sports, and diagnosing other health conditions and diseases related to hCG abnormalities is disclosed here.

This invention can be used to detect extremely low levels of hCG in urine, blood, saliva, or other body fluid for pregnancy test, detection of hCG doping in sports, or diagnosis of other diseases and conditions related to hCG abnormalities so that extremely early pregnancy can be detected and extremely low level hCG doping in sports can be found. This invention can also be used to detect with high sensitivity ectopic pregnancy, spontaneous abortion, hydatiform mole, choriocarcinoma, germ cell tumors, testicular cancer, seminoma, teratoma with elements of choriocarcinoma, islet cell tumor, and other diseases and conditions related to hCG abnormalities.

The materials, procedures, and device models may vary according to the precise method being contemplated and should not be construed as limiting the invention in any way. For example, a mobile material such as mobile Ab1-marker or mobile hCG or its derivative-marker can be already in the place or well. It also can be in a pool and be released to the place or well if needed.

### EXAMPLES

The invention is further described in detail by reference to the following experimental examples. These examples are provided for purposes of illustration only, and are not intended to be limiting unless otherwise specified. Therefore, the invention should in no way be construed as being limited to the following examples, but rather should be construed to encompass any and all variations which become evident as a result of the teaching provided herein.

The materials and methods are now discussed
Urine, blood, saliva, or other body fluid from a woman, an athlete, a patient, or any person
One model of our diagnostic detection device and method, i.e. device 1. See FIG. 1
Another model of our diagnostic detection device and method, i.e. device 2. See FIG. 2
A 3^{rd} model of our diagnostic detection device and method, i.e. device 3. See FIG. 3
A 4^{th} model of our diagnostic detection device and method, i.e. device 4. See FIG. 4
A 5^{th} model of our diagnostic detection device and method, i.e. device 5. See FIG. 5

The results of the experimental examples in theory are now discussed

Improved sensitivity for hCG or its derivative in urine, blood, saliva, or other body fluid for pregnancy test, detection of hCG doping in sports, or diagnosis of other diseases and conditions related to hCG abnormalities can be achieved as shown below.

For device 1 (FIG. 1), if urine, blood, saliva, or other body fluid contains hCG or its derivative (particularly beta subunit of hCG), the detection system can detect the specific marker or change of conditions of the specific marker in the center of the absorbent material, i.e. a positive result. If urine, blood, saliva, or other body fluid does not contain hCG or its derivative (particularly beta subunit of hCG), the detection system cannot detect the specific marker in the center, i.e. a negative result.

For device 2 (FIG. 2), if urine, blood, saliva, or other body fluid contains hCG or its derivative (particularly beta subunit of hCG), the detection system can detect the specific marker or change of conditions of the specific marker in the center of the absorbent material, i.e. a positive result. The detection system may also detect the specific marker or change of conditions of the specific marker in the top of the absorbent material, which indicates there is an excess of marker labeled mobile anti-hCG antibody bound with hCG or its derivative, or marker labeled mobile anti-hCG antibody only. If urine, blood, saliva, or other body fluid does not contain hCG or its derivative (particularly beta subunit of hCG), the detection system cannot detect the specific marker in the center, i.e. a negative result. But the detection system can detect the specific marker or change of conditions of the specific marker in the top of the absorbent material, which indicates the test functions properly and is used as a control for negative result.

For device 3 (FIG. 3), if urine, blood, saliva, or other body fluid contains hCG or its derivative (particularly beta subunit of hCG), the detection system can detect the specific marker or change of conditions of the specific marker in the well after rinse, i.e. a positive result. If urine, blood, saliva, or other body fluid does not contain hCG or its derivative (particularly beta subunit of hCG), the detection system cannot detect the specific marker in the well after rinse, i.e. a negative result. If an immobile anti-hCG antibody conjugated with a specific marker and a mobile anti-hCG antibody are used, and specimen contains hCG or its derivative, change of conditions of specific marker will be detected in the well after rinse. Since a sandwich complex is formed. If specimen does not contain hCG or its derivative, no change of conditions of specific marker will be detected after rinse.

For device 4 (FIG. 4), if urine, blood, saliva, or other body fluid contains hCG or its derivative (particularly beta subunit of hCG), the detection system may detect change of conditions of the specific marker in the well, i.e. a positive result. If urine, blood, saliva, or other body fluid does not contain hCG or its derivative (particularly beta subunit of hCG), the detection system may not detect change of conditions of specific marker in the well, i.e. a negative result. If an immobile anti-hCG antibody and specimen (which may or may not contain hCG or its derivative) conjugated with a specific marker are used, and specimen contains hCG or its derivative, the detection system can detect the specific marker or change of conditions of the specific marker in the well after rinse. If specimen does not contain hCG or its derivative, the detection system cannot detect the specific marker in the well after rinse.

For device 5 (FIG. 5), if urine, blood, saliva, or other body fluid contains hCG or its derivative (particularly beta subunit of hCG), the detection system can detect the specific marker at a decreased level or change of conditions of the specific marker in the well, i.e. a positive result. If urine, blood, saliva, or other body fluid does not contain hCG or its derivative (particularly beta subunit of hCG), the detection system can detect the specific marker at the highest level or no change of conditions of the specific marker in the well, i.e. a negative result. In a microarray model of device 5, it is like array CGH or SNP array. If an immobile anti-hCG antibody/a mobile standard hCG or derivative/specimen (which may or may not contain hCG or its derivative) conjugated with a specific marker are used, and specimen contains hCG or its derivative, the detection system can detect the specific marker or change of conditions of the specific marker in the well. If specimen does not contain hCG or its derivative, no specific marker will be detected. If an immobile anti-hCG antibody/a mobile standard hCG or derivative conjugated with a specific marker 1 (e.g. green)/specimen (which may or may not contain hCG or its derivative) conjugated with a specific marker 2 (e.g. red) are used, and specimen contains hCG or its derivative, the ratio of red:green signals will increase and hCG or its derivative in specimen can be quantified according to the amount of standard hCG or its derivative used in the assay. Because hCG or its derivative from specimen (red) will compete against standard hCG or its derivative (green) for a limited amount of anti-hCG antibody. If specimen does not contain hCG or its derivative, after rinse, the ratio of red:green signals will be zero. If a mobile anti-hCG antibody/an immobile standard hCG or derivative conjugated with a specific marker are used, and specimen contains hCG or its derivative, change of conditions of specific marker may be detected in the well. If specimen does not contain hCG or its derivative, no change of conditions of specific marker will be detected.

Microarray models of device 1, 2, 3, 4, and 5 can be used for simultaneous analysis of multiple specimens (e.g. 5, 25, hundreds or even more specimens, like array CGH or SNP array analysis) or daily sequential analysis of specimen (e.g. 5, 25, 365 or even more days). Tiny amount of materials will be needed for analysis and many copies of test models or tiny devices can be made in one small microarray chip.

### CLAIMS

Whereas this invention has been disclosed with reference to specific embodiments, it is apparent that other embodiments and variations of this invention can be devised by other people skilled in the art without departing from the true spirit and scope of this invention. Therefore, the appended claims include all such embodiments and equivalent variations in accordance with the principals of patent law.

We claim:

1. A diagnostic detection device and method of better detection of human chorionic gonadotropin (hCG) or its derivative in urine, blood, saliva, or other body fluid with higher sensitivity for pregnancy test, detecting hCG doping in sports, or diagnosing other diseases and conditions related to hCG abnormalities, said device and method comprising a material capable of transporting specimen (e.g. urine, blood, saliva, or other body fluid) by capillary action, a mobile anti-hCG antibody conjugated with a specific marker, an immobile anti-hCG antibody, and a highly sensitive detection system, i.e. device and method, for the specific marker or change of conditions of the specific marker. An immobile anti-mouse antibody may also be added into the device, i.e. a 2^{nd} way. In a 3^{rd} way, our novel diagnostic detection device and method only comprise sandwich complex technique and a highly sensitive detection system for the specific marker or change of conditions of the specific marker. In a 4^{th} way, our novel diagnostic detection device and method simply comprise an immobile anti-hCG antibody conjugated with a specific marker and a highly sensitive detection system for the specific marker or change of conditions of the specific marker. For the 4^{th} way, an immobile anti-hCG antibody/specimen (which may or may not contain hCG or its derivative) conjugated with a specific marker, and a detection system can also be used. In a 5^{th} way, our novel diagnostic detection device and method comprise an immobile anti-hCG antibody and a mobile standard hCG or derivative conjugated with a specific marker, and the latter competes against hCG or derivative from urine, blood, saliva, or other body fluid for a limited amount of anti-hCG antibody. For the 5^{th} way, an immobile anti-hCG antibody, a mobile standard hCG or derivative, specimen (which may or may not contain hCG or its derivative) conjugated with a specific marker, and a detection system can also be used. Further, an immobile anti-hCG antibody, a mobile standard hCG or derivative conjugated with a specific marker 1, specimen (which may or may not contain hCG or its derivative) conjugated with a specific marker 2 can also be used. Marker 1 and 2 can be green and red. Relative intensities of competitive reaction of the two antigens to antibody, i.e. green or red signal, will be measured after washing off unbound hCG or its derivative. The red:green ratio will be used to quantify hCG or its derivative in specimen. Finally, a mobile anti-hCG antibody, an immobile standard hCG or derivative conjugated with a specific marker, and a detection system may also be used.

2. The device and method of claim 1, wherein said a specific marker is a fluorochrome such as fluorescein [e.g. fluorescein isothiocyanate (FITC)], rhodamine, Texas Red, Cyanine Cy2, Cyanine Cy3, Cyanine Cy5, AMCA, phycoerythrin, and DyLight dyes, used in a way similar to the way used in immunofluorescence staining of cell or tissue.

3. The device and method of claim 1, wherein said a specific marker is an enzyme [e.g. horse radish peroxide (HRP), alkaline phosphatase (AP)], used in a way similar to the way used in immunocytochemistry or immunohistochemistry of cell or tissue.

4. The device and method of claim 1, wherein said a specific marker is a fluorochrome (e.g. a red/orange fluorescent dye, a green fluorescent dye, an aqua fluorescent dye), used in a way similar to the way used in fluorescence in situ hybridization (FISH) to chromosome.

5. The device and method of claim 1, wherein said a specific marker is a fluorescent dye (e.g. a red fluorescent dye, a green fluorescent dye), used in a way similar to the way used in chromosome and genome analysis using microarrays such as microarray-based comparative genome hybridization (CGH), i.e. array CGH.

6. The device and method of claim 1, wherein said a specific marker is a fluorescent dye (e.g. a red fluorescent dye, a green fluorescent dye), used in a way similar to the way used in chromosome and genome analysis using microarrays such as single nucleotide polymorphism (SNP) array.

7. The device and method of claim 1, wherein said a specific marker is a nanomaterial, e.g. a gold nanoparticle (GNP), a silver nanoparticle, and other metal nanoparticle.

8. The device and method of claim 1, wherein said a specific marker is a nanomaterial, e.g. a quantum dot (QD).

9. The device and method of claim 1, wherein said a specific marker is a nanomaterial, e.g. a magnetic nanoparticle/magnetic bead.

10. The device and method of claim 1, wherein said a specific marker is a nanomaterial, e.g. a nano-bio-chip.

11. The device and method of claim 1, wherein said a specific marker is a nanomaterial, e.g. an encapsulated phase change nanoparticle.

12. The device and method of claim 1, wherein said a specific marker is a nanomaterial, e.g. a silica nanoparticle doped with fluorescence resonance energy transfer (FRET).

13. The device and method of claim 1, wherein said a specific marker is a nanomaterial, e.g. a carbon nanotube (CNT).

14. The device and method of claim 1, wherein said a specific marker is a nanomaterial, e.g. a nanowire, such as a silicon nanowire (SiNW), a gold nanowire, and a conducting polymer nanowire.

15. The device and method of claim 1, wherein said a specific marker is a microcantilever. Microfabricated cantilevers bend according to changes in environment or changes on their surface, and this bending is in the nano-meter scale.

16. The device and method of claim 1, wherein said a specific marker is a nanomaterial, e.g. a nanopore, including synthetic, artificial, and protein-based nanopore.

17. The device and method of claim 1, wherein said a specific marker is a glucose or a small molecular weight material.

18. The device and method of claim 1, wherein said a highly sensitive detection system for the specific marker or change of conditions of the specific marker is a corresponding detection system, i.e. device and method, used to detect the specific marker listed in claims 2-17 respectively. It is a common fluorescence image detection system, a fluorescence detection method similar to that used in FISH to chromosome, a method of imaging and analysis of fluorescence similar to that used in array CGH, a method of imaging and analysis of fluorescence similar to that used in SNP array, a smart phone scanning system for remote analysis of the marker, an optical inspection system, optical sensor, photosensor, enzyme/substrate reaction detector, nano magnet, nano needle biosensor, affinity based immunosensor, electrical sensor, quantum tunneling biosensor, currency change detector, pH change detector, wave change detector, resistance change detector, glucose sensor, optoelectronic devices and methods for small molecular weight materials such as organic photoreceptors, organic photovoltaic devices (OPVs), and organic light-emitting diodes (OLEDs), and other devices and methods for detecting small molecular weight materials. This detection system for the marker can be inside the diagnostic detection device containing a material capable of transporting urine, blood, or saliva by capillary action, a mobile anti-hCG antibody conjugated with a specific marker, an immobile anti-hCG antibody, and an immobile anti-mouse antibody or the diagnostic detection device containing a small well with sandwich complex, an immobile anti-hCG antibody, or an immobile anti-hCG antibody and a mobile standard hCG or derivative conjugated with a specific marker, i.e. marker labeled hCG or derivative. This detection system for the marker can also be a separate device from the diagnostic detection device containing a material capable of transporting urine, blood, or saliva by capillary action, a mobile anti-hCG antibody conjugated with a specific marker, an immobile anti-hCG antibody, and an immobile anti-mouse antibody or the diagnostic detection device containing a small well with sandwich complex, an immobile anti-hCG antibody, or an immobile anti-hCG antibody and a mobile marker labeled hCG or derivative.

19. The device and method of claim 1, wherein said the diagnostic detection device and method containing a material capable of transporting specimen (e.g. urine, blood, saliva, or other body fluid) by capillary action, a mobile anti-hCG antibody conjugated with a specific marker, an immobile anti-hCG antibody, an immobile anti-mouse antibody, and a highly sensitive detection system for the specific marker or change of conditions of the specific marker, or a small well with sandwich complex, an immobile anti-hCG antibody conjugated with a specific marker, an immobile anti-hCG antibody/specimen (which may or may not contain hCG or its derivative) conjugated with a specific marker, or an immobile anti-hCG antibody/a mobile standard hCG or derivative conjugated with a specific marker, an immobile anti-hCG antibody/a mobile standard hCG or derivative/specimen (which may or may not contain hCG or its derivative) conjugated with a specific marker, an immobile anti-hCG antibody/a mobile standard hCG or derivative conjugated with a specific marker 1/specimen (which may or may not contain hCG or its derivative) conjugated with a specific marker 2, a mobile anti-hCG antibody/an immobile standard hCG or derivative conjugated with a specific marker, either as one device or separating the detection system from the others as two devices, can also be connected to the internet through cable, Wi-Fi, or wireless networking to transmit signal or signal change of the specific marker or data obtained by the detection system to a remote place for detection, procession, and analysis. The communications can be through wireless transmission, cable connection, or optical communication with each other and also with a remote place.

20. The device and method of claim 1 can be either at a regular size (like the urine or blood pregnancy test device currently at the market or a well of a 96 well plate for ELISA analysis) or at a very small size (like a microarray chip currently used in array CGH or SNP array). For the latter, i.e. microarray model, very small amount of urine, blood, saliva, or other body fluid is needed. Novel microarray chips of miniature detection models containing tiny amount of a material capable of transporting specimen (e.g. urine, blood, saliva, or other body fluid) by capillary action, a mobile anti-hCG antibody conjugated with a specific marker, an immobile anti-hCG antibody, an immobile anti-mouse antibody, or a tiny well with sandwich complex, an immobile anti-hCG antibody conjugated with a specific marker, an immobile anti-hCG antibody/specimen (which may or may not contain hCG or its derivative) conjugated with a specific marker, or an immobile anti-hCG antibody/a mobile standard hCG or derivative conjugated with a specific marker, an immobile anti-hCG antibody/a mobile standard hCG or derivative/specimen (which may or may not contain hCG or its derivative) conjugated with a specific marker, an immobile anti-hCG antibody/a mobile standard hCG or derivative conjugated with a specific marker 1/specimen (which may or may not contain hCG or its derivative) conjugated with a specific marker 2, a mobile anti-hCG antibody/an immobile standard hCG or derivative conjugated with a specific marker, will be used. A novel microarray scanning device will also be used to detect signal or signal change of the specific marker.
